(19) United States
(12) Patent Application Publication
(10) Pub. No.: US 2009/0176857 A1
(43) Pub. Date: Jul. 9, 2009

(54) Use of Organic Compounds

(76) Inventor: Stephane David Levy, Basking Ridge, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/085,796

(22) PCT Filed: Nov. 30, 2006
(86) PCT No.: PCT/EP2006/011488

§ 371 Date: May 30, 2008

(30) Foreign Application Priority Data
Dec. 2, 2005 (GB) 0524668.1

(51) Int. Cl. A61K 31/404; A61P 1/00

(52) U.S. Cl. 514/415

(57) ABSTRACT

The invention relates to a pharmaceutical composition, use, or method in the treatment of Irritable Bowel Syndrome (IBS) characterized by mixed or alternating bowel habits based on a modification of the Rome II criteria (IBS-M), with 5-hydroxytryptamine type-4 receptor agonists (5-HT₄ agonists), in particular tegaserod.

This invention relates to a pharmaceutical composition, use, or method in the treatment of Irritable Bowel Syndrome (IBS) characterized by mixed or alternating bowel habits based on a modification of the Rome II criteria (IBS-M), with 5-hydroxytryptamine type-4 receptor agonists (5-HT₄ agonists), in particular tegaserod.

Irritable bowel syndrome is a chronic medical disorder that significantly impacts patients' lives. However, the impact of medical treatment of IBS is often not clear. Tegaserod, particularly in the form of its hydrogen maleate salt, a 5-HT₄ receptor agonist, has been shown to be safe and effective in the treatment of IBS with constipation (IBS-C) and chronic iodopathic constipation.

The clinical characteristics of patients with IBS and constipation or diarrhea have been well defined. However, up to 30 to 40% of IBS patients have a mixed or alternating bowel pattern. Established criteria provides for distinct signs and symptoms that define subgroups of IBS.

Symptoms can be distinguished for IBS-C patients and those with mixed/alternating bowel habits. For example, patients with IBS-M may experience differences in abdominal pain and discomfort, bowel movement frequency, days with no bowel movements, stool consistency, days with normal stool consistency, days with straining, and days with urgency, compared with patients with IBS-C.

No medical therapy has been demonstrated to be effective in this challenging subset of IBS sufferers, i.e. in patients with IBS-M.

Gastroenterology; Vol 118, (4) Suppl 2 A1204 (2000) reports that tegaserod has a favourable safety and tolerability profile in patients with constipation predominant and alternating forms of IBS. However, there is no efficacy assessment of the compound in this ill-defined study population. The population in the abstract is “a large population of IBS patients” and then stratified into “alternating IBS based on the presence of at least one symptom (loose/watery stools or >4 BMs/day) This is non-specific, since the presence of this additional symptom does not identify the IBS mixed or alternating subgroup and these symptoms are entirely consistent with a classification of IBS with constipation according to the Rome criteria depending on the presence and/or absence of other symptoms. In contrast, the population in the examples of the present patent application is patients with alternating IBS or mixed IBS defined by the Rome criteria. The Rome Criteria specifically define IBS with constipation and IBS with diarrhea based on symptoms. Patients with a mixture of these symptoms and/or alternating symptoms can be specifically identified by an algorithm derived from the Rome Criteria and as produced for inclusion of patients studies in the study relating to the present invention.

Similarly, Am. J. Gastroenterol.; Vol 97, pp 1176-1181 describes the safety and tolerability of tegaserod in patients with irritable bowel syndrome and diarrhea symptoms but does not provide any evidence of the efficacy of the compound in the study.

Gastroenterology; Vol 126 (4) Suppl 2, A644 (2004), describes the use of the mixed 5-HT₄ receptor full agonist and 5-HT₃ receptor antagonist, renzapride, in ‘mixed-symptom (alternating) irritable bowel syndrome.’ The current invention supports efficacy based on 5-HT₄ agonism, whereas it cannot be concluded from the cited reference that any activity of renzapride is attributable to the 5-HT₄ receptor agonist effect of the compound. Furthermore, in this abstract, renzapride did not demonstrate statistically significant improvements in IBS symptoms and therefore failed to demonstrate efficacy in the study population, however defined.

We have now found that a 5-HT₄ receptor agonist improves multiple symptoms in patients with IBS-M. For example, tegaserod, a well-known 5-HT₄ receptor (partial) agonist, has been found to give satisfactory relief of symptoms and a statistically significant improvement in women with IBS-M.

The results of the study underlying the present invention were presented at Digestive Disease Week 2006, after the priority date of the present invention, and were published in Gastroenterol. 2006; 130 (4, Suppl. 2), A26. Discussion of the results of the study has been published e.g. at www.medscape.com/viewarticle/533101 and a full publication of the study is in draft as at the date of filing the present invention.

Whilst formal criteria for IBS-Mixed have recently been published as part of the Rome III process (Longstreth G, Thompson W G, Chey W D, Houghton L, Mearin F, Spiller R. Rome III: Functional Bowel Disorders. Gastroenterology 2006; 130:1480-91), the Rome III criteria were not available at the date of the invention and at the time the clinical study was performed. According to the present study, IBS was defined using the Rome II criteria (Thompson W G, Longstreth G F, Drossman D A, Heaton K W, Irvine E J, Mueller-Lissner SA. C. Functional bowel disorders and D. functional abdominal pain. In: Drossman D A, Talley N J, Thompson W G, Whitehead W E, Corazziari E, eds. Rome II: functional gastrointestinal disorders: diagnosis, pathophysiology, and treatment. 2^{nd} ed. McLean, VA: Degnon Associates, Inc., 2000:351-432). Since the Rome Criteria specify recommendations for defining IBS-C and IBS-D, the definition for IBS-Mixed and/or alternating can easily be defined (Thompson supra.). In the present study IBS-M was defined as those patients who did not fulfill the Rome II criteria for IBS-C or IBS-D but demonstrated symptoms specifically related to irritable bowel syndrome and presented with disordered bowel function.

The term ‘5-HT₄ agonist’ is used herein throughout the patent application as follows:

A ‘5-HT₄ agonist’ is an agent that has an affinity for serotonin type-4 receptors and is able to mimic the stimulating effects of serotonin at this specific cellular receptor as e.g. is useful in the treatment of certain gastrointestinal diseases such as IBS-C (irritable bowel syndrome with constipation) e.g. in woman or chronic constipation. Examples are tegaserod, zacopride, prucalopride, mosapride, norcisapride, E 3620, ABT 224, VI 0134, ATI 7505, and TD 2749.

Accordingly, the present invention provides a method for the treatment of irritable bowel syndrome characterized by mixed or alternating bowel habits in a patient in need of such treatment, which comprises administering an effective amount of a 5-HT₄ agonist to the patient.

The invention further provides the use of a 5-HT₄ agonist for treating irritable bowel syndrome characterized by mixed or alternating bowel habits, and the use of a 5-HT₄ agonist in the preparation of a medicament for the treatment of irritable bowel syndrome characterized by mixed or alternating bowel habits.

Furthermore, the invention provides a pharmaceutical composition for use in the treatment of irritable bowel syndrome characterized by mixed or alternating bowel habits comprising a 5-HT₄ agonist and suitable excipients.

The uses, pharmaceutical compositions and methods of the present invention represent a new therapy for irritable bowel syndrome characterized by mixed or alternating bowel habits.

Thus in particular embodiments the invention provides:

a method for the treatment of irritable bowel syndrome characterized by mixed or alternating bowel habits in a patient in need of such treatment which comprises administering an effective amount of a 5-HT₄ agonist to the patient;

use of a 5-HT₄ agonist for treating irritable bowel syndrome characterized by mixed or alternating bowel habits, and use of a 5-HT₄ agonist in the preparation of a medicament for the treatment of irritable bowel syndrome characterized by mixed or alternating bowel habits; and

pharmaceutical compositions for use in the treatment of irritable bowel syndrome characterized by mixed or alternating bowel habits comprising a 5-HT₄ agonist and excipients. Suitable 5-HT₄ agonist for use in the invention may include (but are not limited to) the following compounds or a pharmaceutically acceptable salt thereof, or any hydrate thereof: tegaserod, zacopride, prucalopride, mosapride, norcisapride, E 3620, ABT 224, VI 0134, ATI 7505, and TD 2749.

Preferably, the 5-HT₄ agonist is tegaserod, more preferably tegaserod hydrogen maleate.

All the 5-HT₄ agonists mentioned above are known from the literature. This includes their manufacture. For example, tegaserod is prepared as described e.g. in U.S. Pat. No. 5,510,353.

The 5-HT₄ agonists (hereinafter referred to as the Agents of the Invention) may be used in the form of an isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The Agents of the Invention can also be used in any salt form or in the form of their hydrates or include other solvents used for their crystallisation.

The Agents of the Invention are preferably used in the form of pharmaceutical compositions that contain a therapeutically effective amount of active ingredient optionally together with or in admixture with inorganic or organic, solid or liquid, pharmaceutically acceptable carriers which are suitable for administration.

The pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

Preferably, the pharmaceutical compositions are adapted to oral or parenteral (especially intravenous, intra-arterial or transdermal) administration.

The particular mode of administration and the dosage may be selected by the attending physician taking into account the particulars of the patient, especially age, weight, life style, activity level. Most preferably, however, the Agents of the Invention is administered orally.

The dosage of the Agents of the Invention may depend on various factors, such as effectiveness and duration of action of the active ingredient, mode of administration, warm-blooded species, and/or sex, age, weight and individual condition of the warm-blooded animal.

The dose—either administered as a single dose (which is preferred) or in several partial doses—may be repeated, for example once, twice or trice daily. In other words, the pharmaceutical compositions may be administered in regimens ranging from continuous daily therapy to intermittent cyclical therapy.

Preferably, tegaserod hydrogen maleate, one of the Agents of the Invention, is administered in doses which are in the same order of magnitude as those used for the treatment of Irritable Bowel Syndrome constipation predominant (IBS-C), e.g. 6 mg twice daily.

The following Example illustrates the invention described hereinbefore.

### EXAMPLE

Evaluation of the effect of tegaserod (T) on multiple symptoms in women with mixed/alternating bowel habits.

METHODS: A randomized, double-blind, placebo-controlled, multicenter study was performed in patients with IBS-C (based on Rome II criteria) and IBS-M (modification of Rome II). The primary efficacy variable was the patients' assessment of satisfactory relief over 4 weeks treatment with T 6 mg bid or Placebo (P). The proportion of patients reporting satisfactory relief of 4 weeks (75% rule) and improvement during each of the 4 weeks in individual IBS symptoms were also assessed. Treatments were compared using a generalized linear model with logit link.

RESULTS: 661 women were randomized (IBS-C 337; IBS-M 324). Baseline symptom assessment clearly distinguished between the IBS-C and the IBS-M cohorts. Statistically significant differences were found between the two cohorts in bowel movement (BM) frequency, stool consistency and straining. T provided statistically significant improvement in satisfactory relief of IBS symptoms over a 4-week treatment (odds ratio 1.75; 95% CI: 1.35, 2.25; p-value<0.001) for the IBS-C and IBS-M cohorts. In the two cohorts, the percentage of patients experiencing satisfactory relief of IBS symptoms (75% rule) was significantly higher for T when compared to P (IBS-C: 43.3% v 28.9%, p=0.008 and IBS-M: 52.3% v 36.3%, p=0.010). T was significantly superior to P with differences between means for weekly BM frequency (IBS-C: T 6.88; P 6.06 and IBS-M: T 9.78; P 8.34), days/week with no BMs (IBS-C: T 2.31; P 2.18 and IBS-M: T 1.39; P 1.68), stool consistency score using a 7-patient scale (IBS-C: T 3.53; P 2.89 and IBS-M: T 3.95; P 3.49) and days/wk with straining (IBS-C: T 3.08; P 3.55 and IBS-M: T 2.42; P 2.87) (all p<0.05). Adverse event discontinuations were low (IBS-C: T 1.2% v P 2.4% and IBS-M: T 2.5% v P 3.6%). Most frequent AEs (with discontinuations) were diarrhea (1.1%), headache (0.6%) and nausea (0.3%). No clinically significant diarrhea or colitis of any type was reported.

CONCLUSION: This study demonstrates that T is effective and safe in treating the overall symptoms of IBS in patients with a mixed or alternating bowel habit.

1-3. (canceled)

4. A method of preventing or treating irritable bowel syndrome characterized by mixed or alternating bowel habits, comprising administering to a subject in need thereof an effective amount of a 5-HT₄ agonist.

5. The method of claim 4, wherein the 5-HT₄ agonist is tegaserod, zacopride, prucalopride, mosapride, norcisapride, E 3620, ABT 224, VI 0134, ATI 7505, and TD 2749 or pharmaceutically acceptable salts thereof.

6. The method of claim 4, wherein the 5-HT₄ agonist is tegaserod hydrogen maleate.
